(19) United States
(12) Patent Application Publication
(10) Pub. No.: US 2016/0235614 A1
(43) Pub. Date: Aug. 18, 2016

(54) INTEGRATED CRYOTHERAPY APPARATUS

(71) Applicants: Neil Kay, Torrance, CA (US); Eric Levy, Torrance, CA (US)

(72) Inventors: Neil Kay, Torrance, CA (US); Eric Levy, Torrance, CA (US)

(21) Appl. No.: 15/059,614

(22) Filed: Mar. 3, 2016

Related U.S. Application Data
(63) Continuation-in-part of application No. 13/964,750, filed on Aug. 12, 2013, now abandoned.

(51) Int. Cl. A61H 1/00; A61F 5/01

(52) U.S. Cl. CPC A61H 1/008; A61F 5/0109; A61H 2201/0214; A61H 2201/0242; A61H 2201/1645; A61H 2201/5038; A61H 2201/5043; A61H 2205/102

(57) ABSTRACT

The embodiments shown provide an integrated cryotherapy apparatus to alleviate minor pain while preventing skin and soft tissue damage to the affected area of the body. The apparatus includes a flexible outer sleeve configured to releasably house a portion of the containing element and releasably secure the apparatus to the user's body while ensuring uniform compression and application. Further, an integrated cap includes a liquid crystal display and countdown timer to ensure the prevention of soft tissue damage from excessive icing/cooling.

### CROSS-REFERENCE TO RELATED APPLICATION

This Continuation-in-Part (CIP) Application claims priority to U.S. patent application Ser. No. 13/964,750 entitled Ice Therapy System (confirmation no. 2103) filed on Sep. 1, 2013 by Neil J. Kay.

### FIELD

The embodiments shown herein relate to an integrated cryotherapy apparatus, and in particular to an integrated cryotherapy apparatus which alleviates minor aches and pains to a plurality of body parts while preventing the damaging effects of excessive icing/cooling.

### BACKGROUND

Cryotherapy and cold compression are commonly used for the management of pain and inflammation associated with acute injuries, surgical procedures, and orthopedic treatment. The primary reason for cryotherapy in acute pain management is to reduce the soft tissue metabolic rate, helping damaged tissue survive.

However, continuous cold therapy has led to excessive cooling/icing times, which have led to severe skin and tissue damage including frostbite and amputation. Further, it has been shown that vasoconstriction occurs within 10 minutes of traditional cooling devices under 40 degrees Fahrenheit, limiting the supply of oxygen and nutrients in the blood.

Although there are several references to cold compression wraps and refreezable gels, there is not a single prior art reference that discloses a device to provide cryotherapy while eliminating the dangers of excessive icing.

### SUMMARY OF THE INVENTION

Embodiments described herein include an integrated cryotherapy apparatus that provides minor pain alleviation using cold therapy while preventing the dangers associated with excessive icing/cooling. The apparatus further includes a flexible outer sleeve, containing element, and cap with an integrated liquid crystal display (LCD) and suspendable countdown timer. The embodiments enable a user to fill the containing element with a plurality of cooling mixtures, hermetically seal the mixture within the containing element, place the cooled containing element over the affected area, and releasably attach the containing element to an area of the body and secure in place using the flexible outer sleeve. Further, the apparatus enables both uniform compression and cooling to the affected area using a plurality of attachment means located about the periphery of the flexible outer sleeve. The cap further includes an integrated liquid crystal display and countdown timer, which allow the user to input a desired icing time and start a timer, which ensures the user is apprised of the expected ice time and prevents tissue damage associated with excessive icing/cooling.

Other aspects, advantages, and novel features of the embodiments shown herein will become apparent from the following detailed description in conjunction with the drawings.

### BRIEF DESCRIPTION OF THE DRAWINGS

A more complete understanding of the embodiments shown, and the attendant advantages and features thereof, will be more readily understood by reference to the following detailed descriptions when considered in conjunction with the accompanying drawings wherein:

FIG. 1 is an exploded view of the integrated cryotherapy apparatus;

FIG. 2 is a view of the apparatus detached from a user with the cap removed;

FIG. 3 is a perspective view of the containing element and cap;

FIG. 4 is a view of the apparatus releasably attached to a user; and

FIG. 5 is a view of the cap.

### DETAILED DESCRIPTION

The specific details of a single embodiment or variety of embodiments described herein are set forth in this application. Any specific details of the embodiments are used for demonstration purposes only, and no unnecessary limitations or inferences are to be understood therefrom.

The embodiments shown relate to an integrated cryotherapy apparatus. The embodiments shown demonstrate an apparatus that provides minor pain alleviation by releasably attaching the apparatus to the affected area for a predetermined period of time and further alleviating any soft tissue or skin damage associated with excessive icing/cooling.

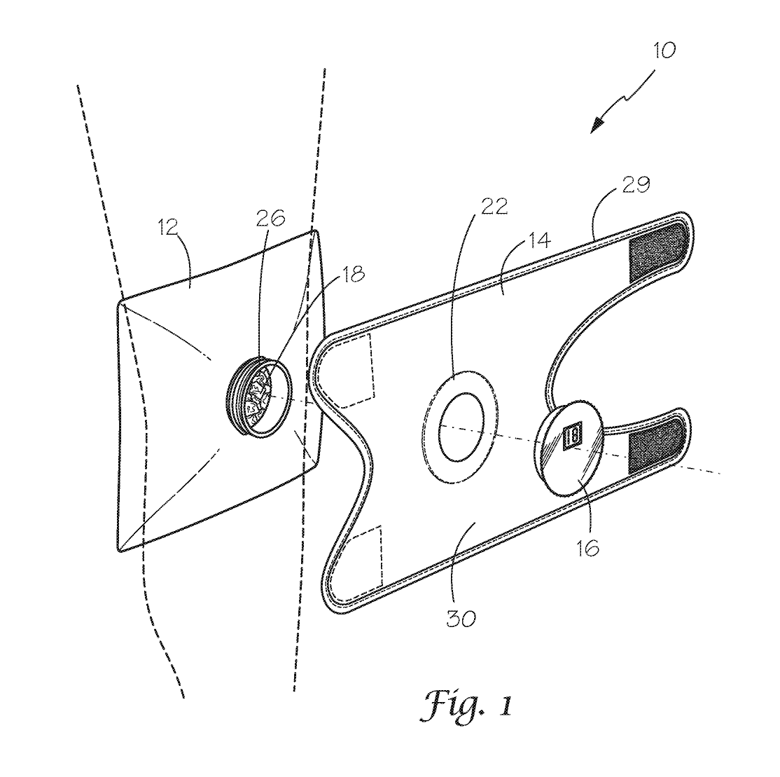

Referring now to the drawings wherein like reference numerals designate identical or corresponding parts throughout the views. There is shown in FIG. 1 an exploded view of the integrated cryotherapy apparatus 10. The apparatus 10 includes a containing element 12, outer “compression” sleeve 14, and cap 16 further including a liquid crystal display and suspendable countdown timer. The apparatus 10 further includes an interior portion 18 which may be filled with a plurality of cooling mixtures, and an exterior portion 20 configured to be releasably secured onto the user. It is contemplated that the interior portion 18 and exterior portion are composed of a single flexible material such as neoprene, rubber, or a plastic material. However, it should be understood that any suitable material may be used as long as it is flexible and capable of holding a filler material.

The containing element 12 is a substantially elliptical collapsible container that is configured to releasably store a plurality of cooling material. Further, the containing element has an opening 22 to enable the user to fill/empty the containing element 12. The opening 22 is dimensioned to enable the user to insert a plurality of cooling materials such as water, ice, frozen gels, or any other material capable of being cooled and inserted within the interior portion 18. The substantially elliptical configuration of the containing element 1 enables the user to fill the interior portion 18 and ensure the exterior portion 20 is a uniform temperature before actuating a countdown timer 24. The containing element contains a plurality of flexible sidewalls to optimally contour the containing element around the body part of the user. Further, the flexible sidewalls help facilitate the freeflow of cooling mixture within the containing element to help maintain a uniform temperature on the exterior surface of the containing element, which is in communication with a body part of the user. Further, the opening 22 enables the user to fill to a desired level before any treatment or the countdown timer 24 is actuated. The embodiments provide a contoured interior portion 18 that is safer than current designs incorporating a “jet system,” which provides for non-uniform cooling, which has been shown to cause tissue damage and even frostbite. Further shown in FIG. 1 is a sealable edge 26 located at the top of the containing element 12 which is releasably secured with the cap 16 when engaged, to create a hermetic seal to the interior portion 18.

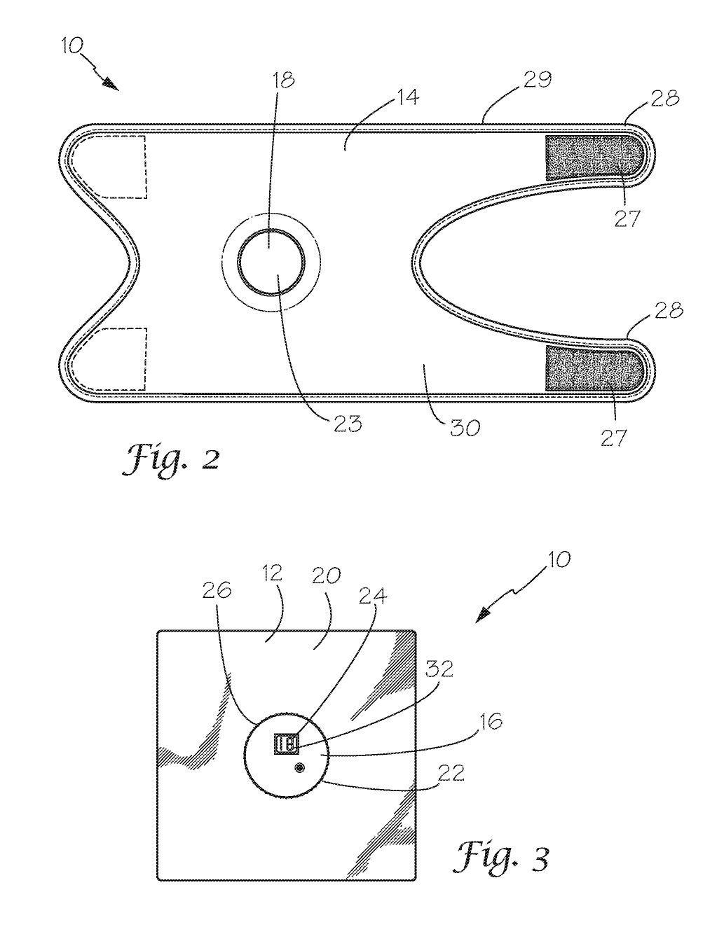

Referring now to FIG. 2, a view of the compression sleeve 14. The compression sleeve 14 may be made of a flexible synthetic polymer, rubber, or similar material, and is configured to releasably secure the containing element 12 using a plurality of attachment means 27 located along the periphery of the outward extending extremities 28 onto the user's body. The compression sleeve 14 further includes an sleeve interior portion 29 which is in contact with both containing element 12 and the user's body part, and an sleeve exterior portion 30. Further, the compression sleeve 14 is designed to be flexed and stretch to fit a plurality of body parts, and may stretch to fit the intended body part of the user, and provide a more secure fit, and uniform compression to the desired body part. The compression sleeve may be adjustable in size, or interchangeable, and intended to fit a knee, elbow, back, or other part of the body that the user desires.

Referring now to FIG. 3, a view of the containing element 12 and cap 16, The cap 16 further includes an integrated liquid crystal display (LCD) 32 and countdown timer 24. The liquid crystal display 32 further includes a plurality of light emitting diodes (LED) 34 which provide for local dimming and high resolution images while simultaneously reducing the load on integrated power supply. The liquid crystal display 32 and countdown timer 24 enable the user to set a desired cooling period, actuate a timer, and ensure that overicing is prevented. Further, the countdown timer 24 may be suspended by the user for a pre-determined period, which enables the user to ensure the exterior portion 20 is a uniform temperature before the countdown is resumed. Further, the countdown timer 24 counts down in seconds, minutes, and hours. The countdown timer 24 stops at at zero, and further includes time zone support. The countdown timer 24 may display time as “4” to indicate 4 minutes, or may display “1 d 2 h 7 m” as one day, two hours, and seven minutes, and further includes a memory module that recognizes future date and time combinations and may accept command line arguments to create batch files for most frequently used countdown values. The liquid crystal display 32 allows the user to suspend (i.e., pre-determined break times) and may include a ticking background sound. Also, the countdown timer 24 includes a “countdown bar” which slowly fills as the time is counted down and the “countdown bar” is completely tilled when the time has exprired to provide visual orientation for the user.

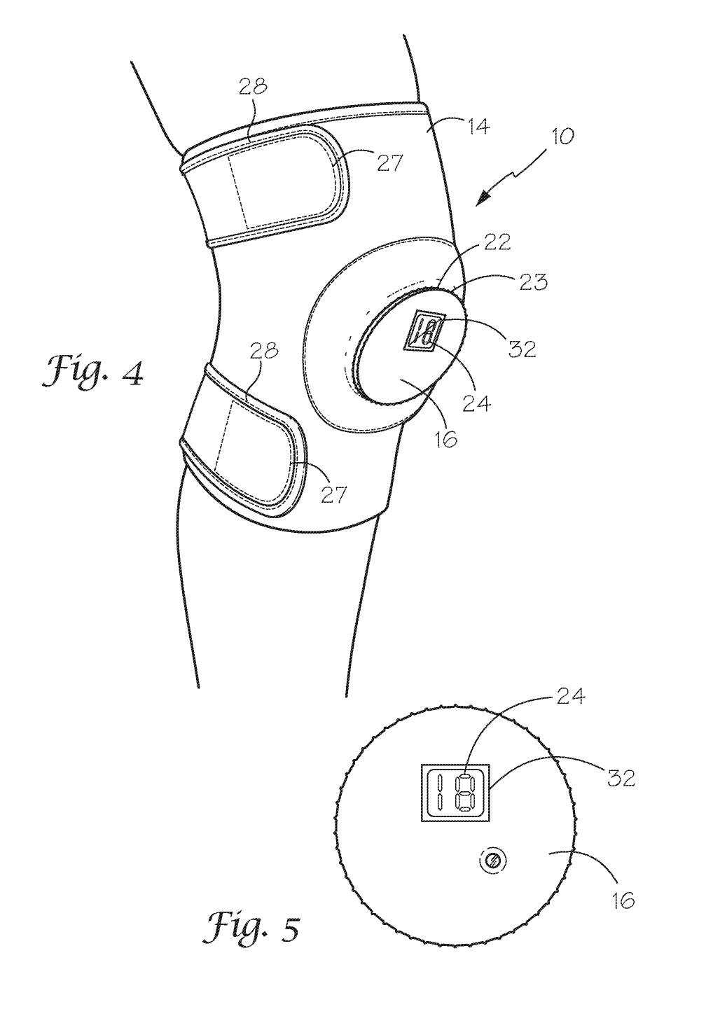

Referring now to FIG. 4, a view of the apparatus 10 releasably secured to the knee of the user using the plurality of attachment means 27. The compression sleeve 14 ensures the apparatus 10 remains in place and uniform compression is applied to the affected area. The cap 16 is applied to provide a hermetic seal to the interior portion 18 and the LCD 32 and countdown timer 24 are displaying a generic time of “18” to indicate minutes left until the end of the cryotherapy session.

Referring now to FIG. 5, a view of the cap 16 and LCD 32. The LCD 32 allows an audio/visual message to be displayed to the user during the countdown, run a program, or change the display font if necessary.

Persons skilled in the art will appreciate that the present embodiments go beyond what is particularly shown and described hereinabove. In addition, note that all of the accompanying drawings are not to scale unless otherwise noted. A variety of modifications and variations are possible in light of the above description without departing from the following claims.

What is claimed is:

1. An integrated cryotherapy apparatus to provide optimal pain alleviation while preventing the damaging effects of excessive icing/cooling, the apparatus comprising:
a flexible compression sleeve further including a plurality of attachment means configured to provide a uniform compression onto a body of the user;
a containing element dimensioned to fit within a portion of the flexible outer sleeve and configured to releasably store at least a cooling mixture;
a cap further including an integrated liquid crystal display and suspendable countdown timer to enable a user to input a plurality of incremental countdown times.

1. The apparatus of claim 1, wherein the flexible compression sleeve is composed of a low durometer elastic material to enable uniform contouring over an area of a user's body while releasably attached.

2. The apparatus of claim 2, wherein the flexible compression sleeve which further contains at least one outward extending extremity configured to be releasably attached over an area of the users body.

4. The apparatus of claim 3, wherein the at least one outward extending extremity further contains an attachment means.

5. The apparatus of claim 4, wherein the flexible compression sleeve further include an opening dimensioned to enable a user to access the cap while releasably attached over the area of the users body.

6. The apparatus of claim 1, wherein the containing element further includes a plurality of flexible sidewalls to provide continuous circulation of the cooling mixture.

7. The apparatus of claim 6, wherein the containing element is composed of conductive thermal exchange material to provide a uniform temperature.

8. The apparatus of claim 1, wherein the liquid crystal display and countdown timer enable the user to suspend the countdown timer for a predetermined period of time.

8. The apparatus in claim 1, wherein the liquid crystal display further includes a plurality of light emitting diodes which are configured to display at least a remaining time on the countdown timer, a time of day, a time zone, and a countdown bar.

9. An integrated cryotherapy apparatus, the apparatus comprising:
a substantially elongated flexible compression sleeve further including a plurality of outward extending extremities configured to be releasably attached to a user body and provide a uniform compression;
a substantially elongated containing element dimensioned to fit within a portion of the flexible outer sleeve and configured to provide a uniform cooling exchange to the user's body; and
a substantially elliptical cap further including a planar integrated liquid crystal display and a countdown timer configured to enable a user to selectably suspend a countdown.

10. The apparatus of claim 9, wherein the flexible compression sleeve is composed of an elastic material to provide a uniform application to an area of the user while releasably attached.

11. The apparatus of claim 9, wherein the containing element further includes a plurality of flexible sidewalls dimensioned to provide a continuous uniform cooling effect to an affected area of a body.

12. The apparatus of claim 11, wherein the containing element is composed of a rubber or polymer material to provide a uniform temperature exchange over the affected area of the body.

13. The apparatus of claim 9, wherein the cap is further configured to provide a hermetic seal to a cooling mixture releasably housed within an interior portion of the containing element.

14. The apparatus of claim 13, wherein the liquid crystal display within the cap is configured to display at least a day/hour/second incremental countdown.

15. The apparatus of claim 14, wherein the countdown timer further includes an audio alarm to provide a notification when the countdown timer has expired.

16. The apparatus of claim 9, wherein the liquid crystal display further includes a countdown bar configured to provide a further visual notification of a remaining time on the countdown timer.

17. The apparatus of claim 16, wherein the countdown timer further includes a memory module configured to store at least one previous therapy session.

18. An integrated cryotherapy apparatus to prevent excess icing and skin/tissue damage, the apparatus comprising:
a substantially elliptical compression sleeve in fluid communication with a containing element configured to provide a grip a body of a user while releasably attached;
a containing element reliably housed within the substantially elliptical compression sleeve while releasably attached further including a plurality of flexible sidewalls configured to hermetically contain at least a cooling mixture at a time;
a substantially cylindrical cap further including a planar facing containing a display and suspendable countdown timer configured to notify a user when an icing session has expired.
